(19) United States
(12) Patent Application Publication
(10) Pub. No.: US 2010/0055047 A1
(43) Pub. Date: Mar. 4, 2010

(54) Methods for treating bronchial premalignancy and lung cancer

(76) Inventors: Yiyu Zou, Bronx, NY (US); Roman Perez-Soler, New York, NY (US)

Correspondence Address: AMSTER, ROTHSTEIN & EBENSTEIN LLP, 90 PARK AVENUE, NEW YORK, NY, 10016, US

(21) Appl. No.: 12/583,642

(22) Filed: Aug. 24, 2009

Related U.S. Application Data
(60) Provisional application No. 61/190,097, filed on Aug. 26, 2008.

(51) Int. Cl. A61K 9/12; A61K 31/706; A61P 35/00

(52) U.S. Cl. 424/45; 514/43

(57) ABSTRACT

The present invention is directed to methods for treating bronchial premalignancy and/or lung cancer in a subject comprising administering to the subject an aerosolized pharmaceutical composition comprising a therapeutically effective amount of 5-azacytidine and a pharmaceutically acceptable carrier. The present invention is also directed to methods for decreasing the likelihood of occurrence of bronchial premalignancy and/or lung cancer in a subject comprising administering to the subject an aerosolized pharmaceutical composition comprising a prophylactically effective amount of 5-azacytidine and a pharmaceutically acceptable carrier. The present invention is also directed to pharmaceutical compositions for treating bronchial premalignancy and/or lung cancer in a subject comprising a therapeutically effective amount of 5-azacytidine and a pharmaceutically acceptable carrier. The present invention is also directed to pharmaceutical compositions for decreasing the likelihood of occurrence of bronchial premalignancy and/or lung cancer in a subject comprising a prophylactically effective amount of 5-azacytidine and a pharmaceutically acceptable carrier.

### CROSS REFERENCE TO RELATED APPLICATION

This application is a U.S. Utility Application claiming the benefit of U.S. Provisional Application No. 61/190,097, filed Aug. 26, 2008, the content of which is hereby incorporated by reference in its entirety.

### FIELD OF THE INVENTION

The present invention generally relates to methods for treating or preventing bronchial premalignancy and/or lung cancer in a subject by the administration of an aerosolized pharmaceutical composition comprising a therapeutically or prophylactically effective amount of 5-azacytidine and a pharmaceutically acceptable carrier.

### BACKGROUND OF THE INVENTION

Throughout this application various publications are referred to by Arabic numerals in parentheses. Full citations for these references may be found at the end of the specification immediately preceding the claims. The disclosures of these publications are hereby incorporated by reference in their entireties into the subject application to more fully describe the art to which the subject application pertains.

Lung cancer is the number one cause of cancer-related death, causing an estimated one million deaths worldwide annually (1). Lung cancer originates in the bronchial epithelium as a result of cumulative genetic damage due to the exposure of tobacco carcinogens in at least 80% of the cases. Bronchial premalignancy is not confined to a single area of the bronchial tree; it tends to be multifocal, and as a result cannot be treated by local direct injection of the therapeutic agent. Most primary tumors tend to grow as a solitary mass except in the case of bronchial alveolar carcinoma (BAC) where the primary tumor may grow and disseminate endobronchially in many cases.

One of the mechanisms of carcinogenesis of lung cancer is aberrant methylation of CpG islands in the promoter regions of tumor suppressor genes leading to underexpression or absence of the proteins of those genes thus propagating tumorigenesis (7, 8). CpG islands are methylated in 3 situations; (a) inactive X chromosome (9), (b) gene imprinting (10), and (c) in tumors (11, 12).

Of the tumor suppressor genes, p16^{INK4a} (p16) has been one of the most extensively studied. The p16 gene is inactivated in >70% of cell lines derived from all histologic subtypes of NSCLC (13, 14) through homozygous deletion or aberrant promoter region hypermethylation (15). The strongest evidence supporting early methylation of p16 is the observation that methylation of this gene can precede clinical diagnosis of lung cancer (16).

Another tumor suppressor gene is Death-Associated Protein (DAP) kinase gene that encodes for a Ca^{2+/} calmodulin-regulated serine/threonine kinase. It is actively involved in interferon-alpha, tumor necrosis factor-alpha (TNF-α), or Fas-ligand induced apoptosis (17, 18, 19). Hypermethylation of DAP kinase promoter directly reduces the sensitivity to tumor necrosis factor-related apoptosis-inducing ligand (TRAIL)-induced apoptosis in human NSCLC cell lines (20, 21), and may result in downregulation of p53 responsive genes. Treatment of TRAIL-resistant NSCLC cell lines with demeythylation agents such as 5-aza-2′-deoxycytidine could reexpress DAP kinase and sensitize the cells to TRAIL.

The cadherins, important adhesion molecules family genes, are also frequently methylated. E-cadherin, the prototype cadherin family member and an important tumor suppressor gene, helps in controlling cell growth and differentiation by activating internal signaling (22, 23). Loss of E-cadherin-mediated cell-cell adhesion contributes to the transition from benign, non-invasive tumors (adenoma) to malignant, invasive tumors (carcinoma) (28). Besides the genetic impairment of E-cadherin-mediated cell adhesion caused by deletion or mutation, hypermethylation also directly suppresses E-cadherin promoter activity in invasive carcinoma cells (24, 25). In some tumor types, inactivation of E-cadherin function by hypermethylation appears to be a major oncogenic mechanism. For example, hypermethylation of E-cadherin promoter was found in 83% of examined patients with papillary thyroid carcinoma (26) and in 34% of resected tumor tissues from NSCLC patients (27). The frequent loss of E-cadherin mediated cell adhesion in epithelial cancers, together with its function as a repressor of tumor progression, suggests that E-cadherin is an important tumor suppressor gene (28).

Similar to p16, DAP kinase and E-cadherin, the expression of most tumor suppressor genes can be down regulated due to hypermethylation. Thus, it makes DNA-methyl transferase inhibitors like 5-azacytidine and 5aza-2′-deoxycytidine potential therapeutic agents to cause hypomethylation or demethylation and re-expression of these genes; i.e. demethylation, can serve as an important method to activate the function of tumor suppressor genes.

5-azacytidine (5Aza) is currently approved by the Food and Drug administration for the treatment of myelodysplastic syndromes (29). Additionally, U.S. Pat. No. 7,250,416 and U.S. Publication No. 2006/0063735 describe compositions comprising 5Aza and its potential in treating certain cancers. Azacytidine is phosphorylated by a series of kinases to azacytidine triphosphate, which is incorporated into RNA, disrupting RNA metabolism and protein synthesis. Azacytidine diphosphate is reduced by ribonucleotide reductase to 5-aza-2V-deoxycytidine diphosphate, which is phosphorylated to triphosphate and incorporated into DNA. It binds stoichiometrically to DNA methyltransferases and causes hypomethylation of replicating DNA (30, 31). Azacytidine is also a cytotoxic agent in proliferating cells, but the concentration of azacytidine required for maximum inhibition of DNA methylation in vitro does not suppress synthesis of replicating DNA (32).

Between 1973 and 1977, there were at least 9 clinical studies of solid tumor patients treated with 5Aza, which included 78 lung cancer patients (37). The patients received 5Aza either intravenously or subcutaneously, but not via aerosol administration. In one of the largest studies, one of 24 lung cancer patients had a partial remission, which was transient. In the other patients, there apparently was no benefit.

More recently, the use of 5Aza in combination with other drugs for the treatment or prevention of cancer has been described (38, 39). In addition, it is believed that Johns Hopkins and the University of Mexico began a Phase I/II study in 2006 of the oral histone deacetylase inhibitor MS-275 in combination with 5Aza (by injection) for the treatment of patients with recurrent advanced Non-Small Cell Lung Cancer.

### SUMMARY OF THE INVENTION

In contrast with the prior art, the present inventors have unexpectedly found that in an animal lung cancer model, an increase in survival time and reduced toxicity was observed in those animals receiving 5Aza via intratracheal injection over those animals receiving 5Aza via intravenous injection or no treatment at all.

In accordance with this discovery, the present invention is directed to a method for treating bronchial premalignancy or lung cancer in a subject comprising administering to the subject an aerosolized pharmaceutical composition comprising a therapeutically effective amount of 5-azacytidine and a pharmaceutically acceptable carrier.

The present invention is also directed to a method for decreasing the likelihood of occurrence of bronchial premalignancy or lung cancer in a subject comprising administering to the subject an aerosolized pharmaceutical composition comprising a prophylactically effective amount of 5-azacytidine and a pharmaceutically acceptable carrier.

In addition, the present invention is directed to a pharmaceutical composition for treating bronchial premalignancy and/or lung cancer in a subject comprising a therapeutically effective amount of 5-azacytidine and a pharmaceutically acceptable carrier.

Lastly, the present invention is directed to a pharmaceutical composition for decreasing the likelihood of occurrence of bronchial premalignancy and/or lung cancer in a subject comprising a prophylactically effective amount of 5-azacytidine and a pharmaceutically acceptable carrier.

### BRIEF DESCRIPTION OF THE FIGURES

FIG. 1. Bar graph indicating aerodynamic size of aero-5aza.

FIG. 2. Graph indicating cytotoxicity of 5aza on human NSCLC cell lines.

FIG. 3. Western blot showing re-expression of E-cadherin and DAPK in human NSCLC cells treated with 5aza.

FIG. 4. Graph showing acute toxicity of intratracheally administered 5aza.

FIGS. 5-1, 5-2 and 5-3. 5-1: Graph indicating dose finding and lethal toxicity of 5aza. 5-2 and 5-3: Graphs indicating antitumor efficacy of intratracheally administered 5aza.

### DETAILED DESCRIPTION OF THE INVENTION

In accordance with the present invention, a therapeutically or prophylactically effective amount of 5-azacytidine is administered to a subject to treat or prevent bronchial premalignancy or lung cancer in the subject.

As used herein, a “therapeutically effective amount” to treat bronchial premalignancy is an amount that inhibits or retards the growth of a bronchial premalignancy, or kills the bronchial premalignant cells, or otherwise prolongs survival of the subject by inhibiting or retarding the growth of the premalignant cancerous cells. A “therapeutically effective amount” to treat lung cancer is an amount that inhibits or retards the growth of lung cancer cells, or kills the lung cancer cells, or otherwise prolongs survival of the subject by inhibiting or retarding the growth of lung cancerous cells.

As used herein, “prophylactically effective amount,” with respect to either bronchial premalignancy or lung cancer, is an amount effective to prevent, reduce, reverse or suppress the occurrence or likelihood of occurrence of premalignant or cancerous cell formation or growth.

In the methods and formulations of the present invention, the subject includes those patients having or diagnosed as having a bronchial premalignancy or lung cancer, as well as those patients that may be predisposed to having bronchial premalignancies or lung cancer (e.g., long term users of smoking tobacco products or individuals exposed to inhaled carcinogenic substances such as asbestos, second hand smoke and radon). The subject also includes those patients that have been diagnosed and potentially treated with chemotherapeutics and/or radiation, who are then subjected to treatment with a therapeutic or prophylactic dose of aerosolized 5-azacytidine to treat or prevent the reoccurrence of any tumors or any potentially lingering tumors that have not been previously treated.

As used herein, the lung cancer that can be treated or prevented in accordance with present invention includes (1) small cell, (2) non small cell, (3) mixtures of small cell and non small cell cancers, (4) sarcomas, or (5) lymphomas. With respect to small cell lung cancer, the cancer may be small cell carcinoma, mixed small/large cell carcinoma or combined small cell carcinoma. With respect to non-small cell lung cancer (NSCLC), the cancer may be squamous cell carcinoma, adenocarcinoma and large cell undifferentiated carcinoma. Preferably, the lung cancer is NSCLS. As used herein, bronchial premalignancy includes those cells that may or have a propensity to differentiate into lung cancer cells.

In accordance with the present invention, 5-azacytidine is administered in an aerosolized formulation. Pharmaceutically acceptable carriers suitable for aerosolized formulation include, but are not limited to, saline, phosphate buffered saline, Ringer's solution, lactated Ringer's solution, Locke-Ringer's solution, Kreb's Ringer's solution, Hartmann's balanced saline solution, and/or heparinized sodium citrate acid dextrose solution. Additionally, U.S. Pat. Nos. 5,376,386, and 5,254,330 which are hereby incorporated by reference into the present application, describe aerosol carriers suitable for inhaled delivery of medicaments to the lung.

In the methods of the present invention, administration is by inhalation of the aerosolized pharmaceutical composition for delivery to lungs or bronchial tissues. Formulations suitable for intrapulmonary or nasal administration have a particle size, for example, in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. In the preferred embodiment of the present invention, the aerodynamic size of the 5-azacytidine is from about 0.1 to about 3 microns in diameter. Suitable formulations also include aqueous or oily solutions of the active ingredient. Methods for the inhaled delivery of pharmaceutical compounds are well known in the art, for example, as those disclosed in U.S. Pat. Nos. 4,044,126, 4,414,209, and 4,364,923. These patents are hereby incorporated by reference into the subject application. Devices, such as nebulizers and inhalers, for the administration of aerosolized compositions are also well known in the art.

In terms of dosages, it is preferred that the dosage of 5Aza is from 2 mg/m² to 600 mg/m². More preferably, the dosage is from 5 mg/m² to 200 mg/m². Most preferably, the dosage is from 7.52 mg/m² to 30 mg/m².

In the present invention, the active anticancer agent in the pharmaceutical composition is 5-azacytidine. In the preferred embodiment, 5-azacytidine is the only active anticancer agent in the pharmaceutical composition.

In accordance with the present invention, a therapeutically or prophylactically effective amount of a demethylating agent is administered to a subject to treat or prevent bronchial premalignancy or lung cancer in the subject.

As used herein, a “demethylating agent” is any substance that can inhibit methylation, resulting in the expression of previously hypermethylated silenced genes. Numerous demethylating agents are known in the art. The most commonly known demethylating agents are cytidine analogs, which include but are not limited to, 5-azacytidine, 5-azadeoxycytidine (decitibine), 5-fluorodeoxycytidine, cytarabine, gemcitabine, and pseudoisocytidine.

In one embodiment, the demethylating agent is decitibine. In terms of dosages, it is preferred that the dosage of decitibine is from 0.5 mg/m² to 8000 mg/m². More preferably, the dosage is from 1 mg/m² to 5000 mg/m². Most preferably, the dosage is from 2 mg/m² to 2500 mg/m².

This invention will be better understood from the Experimental Details, which follow. However, one skilled in the art will readily appreciate that the specific methods and results discussed are merely illustrative of the invention as described more fully in the claims that follow thereafter.

### Experimental Details

Formulation. The formulation is pharmaceutical grade powder or lyophilized powder of 5-Azacytidine or decitabine. The solid drugs are dissolved into Lactated Ringer's Injection liquid (LRJ) 2-15 min before use. The concentration is 1-25 mg/ml for 5-azacytidine and 1-60 mg/ml for decitabine.

Aerosol equipment. A PARI personal compressor and a LC star nebulizer were used in this study. The pressure should be 20-28 psi, and the aerosol generated rate should be 0.3±0.03 ml/min. Other clinically used aerosol equipment with similar functions can be used as well.

Aerodynamic size. Aerodynamic size directly affects the deposit site of aerosol delivered drug. It has been suggested that droplets <5 μm, particularly <3 μm, deposits were most frequent in the lower airways of human and, thereby, appropriate for pharmaceutical inhalation aerosols in humans (33,34).

The aerodynamic diameters of aerosol droplets of 5-azacytidine were determined with extrusion-precipitation method using a 7-Stage Cascade Impactor (In-Tox Products, Albuquerque, N. Mex.) linked to PARI's personal compressor and LC star nebulizer system. 5-azacytidine solution (4 ml) was aerosolized under the airflow rate of 5 L/min. The condensed aerosol samples were collected at 3 different periods, from 1 to 1.5 min, from 3 to 3.5 min, and from 5 to 5.5 min. Aerodynamic size and fraction of aerosol with a particular size range were measured and calculated as per manufacturer's protocol. The data was mean±SD of the aerodynamic size based on weight (bars) and cumulative weight (line) from 3 independent experiments.

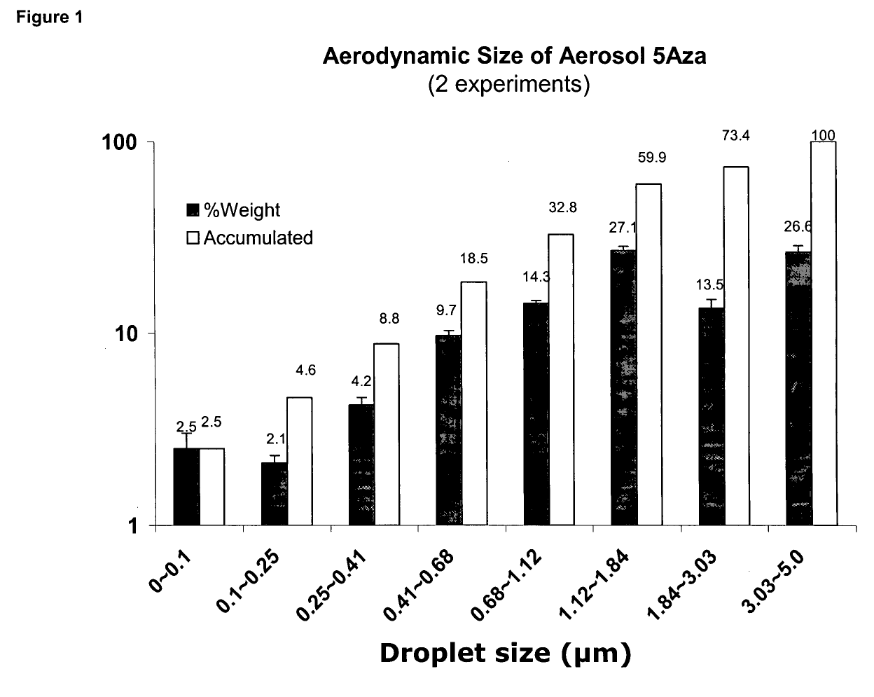

The results shown indicate that under these experimental conditions, the aerodynamic size of 5-azacytidine is about 97% (weight) of droplets measured between 0.2˜3 μm in diameter (FIG. 1).

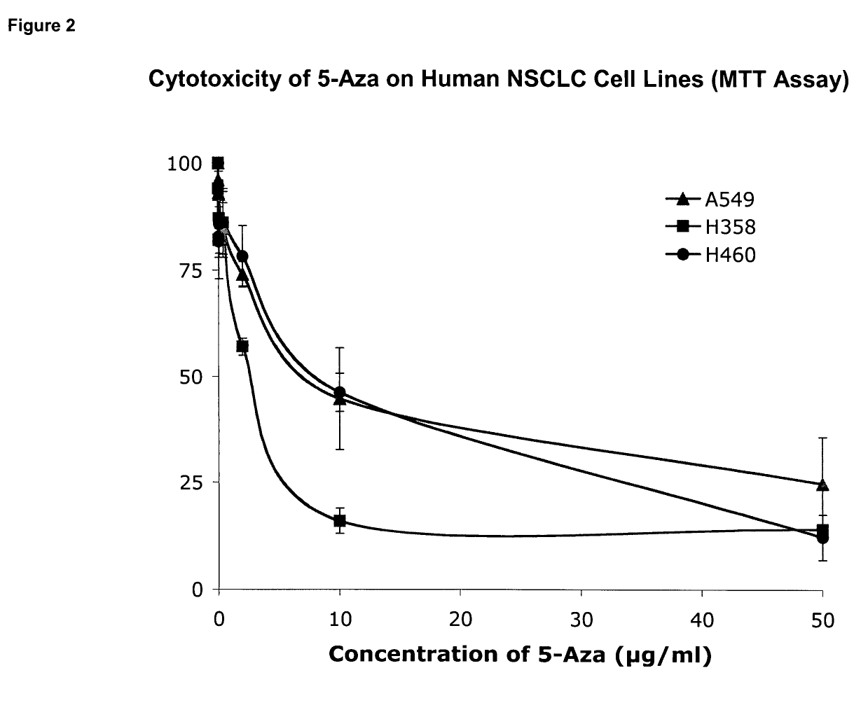

Cytotoxicity of 5-azacytidine. Human NSCLC cell lines H358, H460, and A549 were exposed to different concentrations of 5-azacytidine in a 96-well plate for 3 days. The cytotoxicity was measured by MTT assay. The concentrations inhibiting 50 percent cell growth (Inhibitory concentration 50 or IC₅₀) of 5-azacytidine against these NSCLC cell lines were 4˜14 μg/ml (FIG. 2).

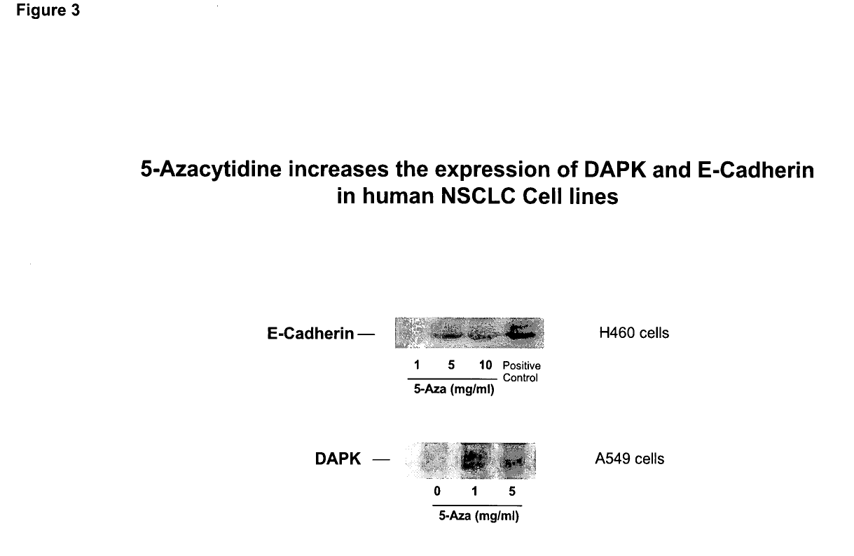

The demethylation function of 5-azacytidine. DAPK and E-cadherin are important tumor suppressor proteins. They are not detectable by Western blotting assay in the parental human NSCLC cell lines A549 and H460 because of methylation. After exposing this cell line to different concentrations of 5-azacytidine for 24 hours, the expressions of DAPK and E-cadherin were significantly enhanced, and therefore, their levels were detected by Western blotting assay. The effective concentration of 5-azacytidine is 1˜10 μg/ml (FIG. 3).

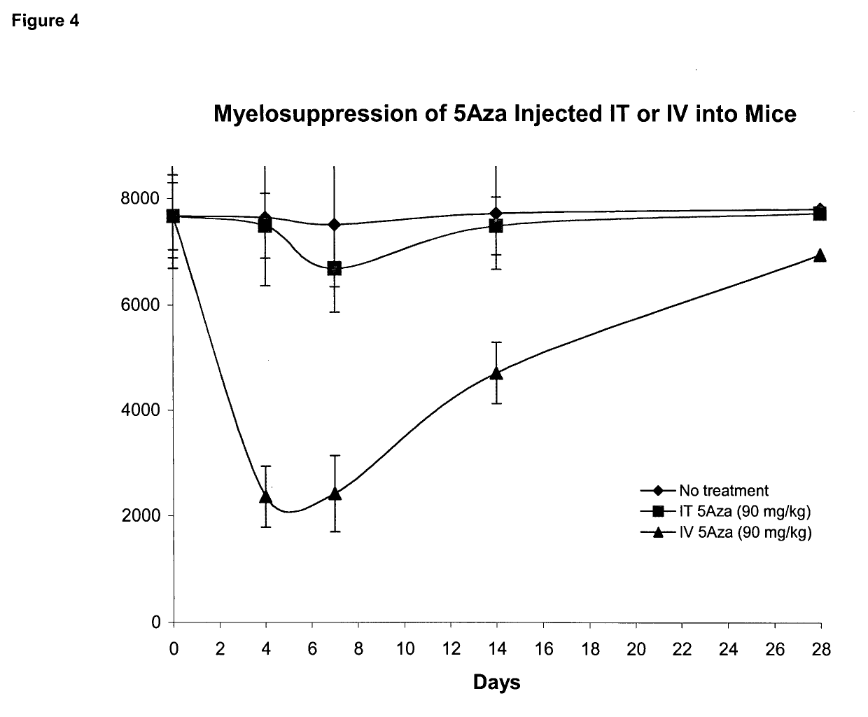

Acute toxicity. The dose limiting toxicity of intravenous 5-azacytidine or decitabine is severe myelosuppression. In order to test whether aerosol or intratracheal injection can reduce the myelotoxicity or alter the toxicity profile, a dose of 90 mg/kg, the maximum tolerated dose (MTD) for intravenous injection (IV) of 5-azacytidine, was used to treat ICR mice by intravenous injection to mimic current therapy. The same dose of intratracheal injection (IT) of 5-azacytidine was used to mimic aerosol administration (35). Complete blood count (CBC), creatinine, liver function tests, and pathological evaluation of H/E staining of lungs, kidney and liver were performed to assess for toxicity differences. The myelotoxicity of intratracheal injected 5-aza was significantly lower than that of IV 5-aza. The highest white blood cell (WBC) reduction in the IT group was 11±1.0% on day 7 as compared to 69±8.3% WBC reduction in the IV group on day 4 (p=0.017, FIG. 4). There were no hepatic or renal toxicities in either arm. IT 5-aza caused reversible lung toxicity that peaked on day 7, reduced on day 14 (Table 1 and 2). The toxicity probably related to the buffer used as vehicle.

TABLE 1
Toxicity grade
of lungs of the mice treated with IT 5 Aza

Day

| | 4 | 7 | 14 | 28 |
|---|---|---|---|---|
| IT Aza | 0 | 1-2 | 0 | 0 |
| IT Vehicle* | 0 | 1-2 | 0 | 0 |
| IV Aza | 0 | 0 | 0 | 0 |
| No Treat | 0 | 0 | 0 | 0 |

*where vehicle is Lactated Ringer's Injection used to dissolve 5Aza

TABLE 2
Explanation of the toxicity grade

| | Toxicity grade | | | | |
|---|---|---|---|---|---|
| | 0 | 1 | 2 | 3 | 4 |
| % Involved tissue | 0 | 0~10 | 10~30 | 30~60 | >60 |
| Severity | No | Mild | Moderate | Severe | Life threaten |

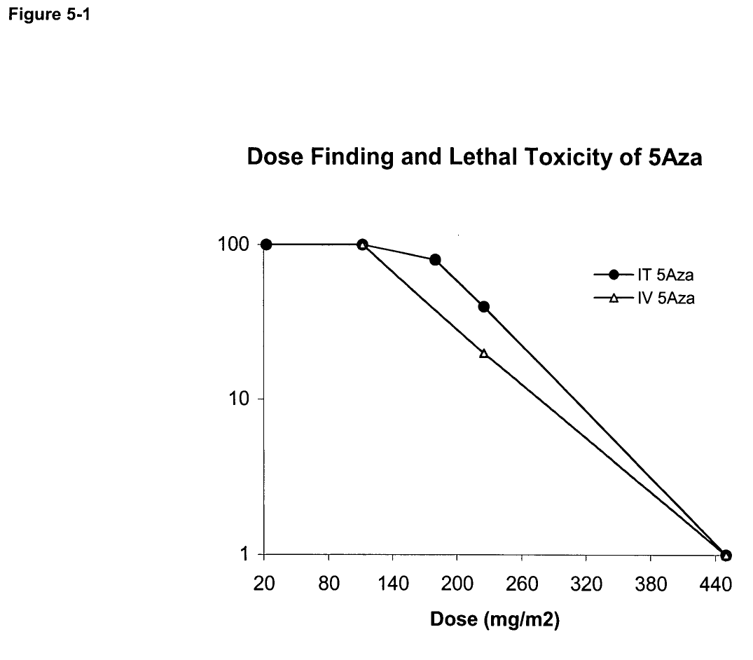

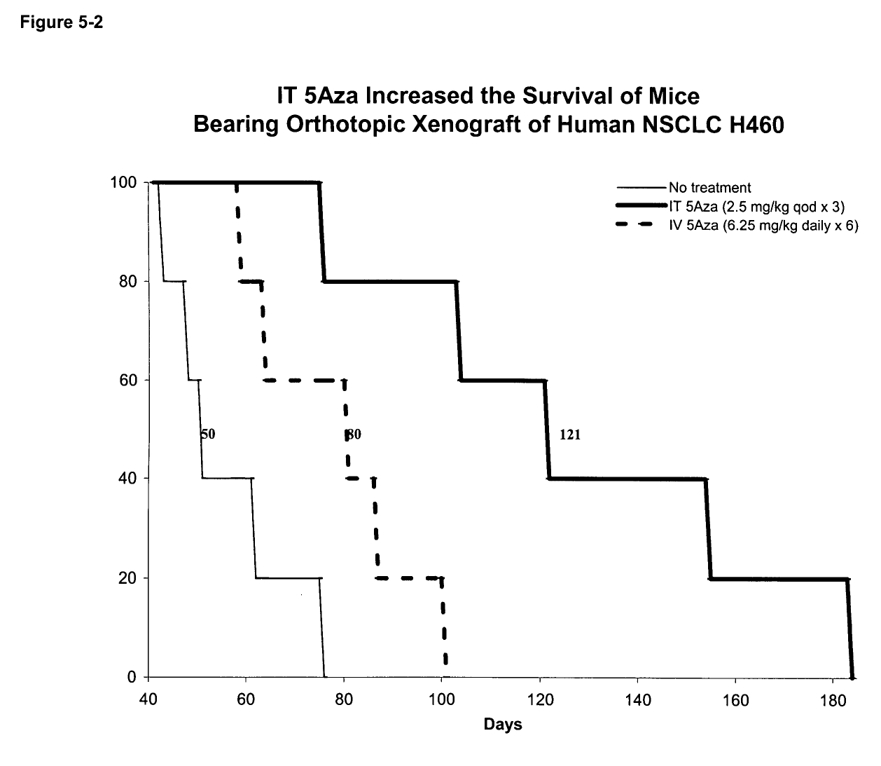

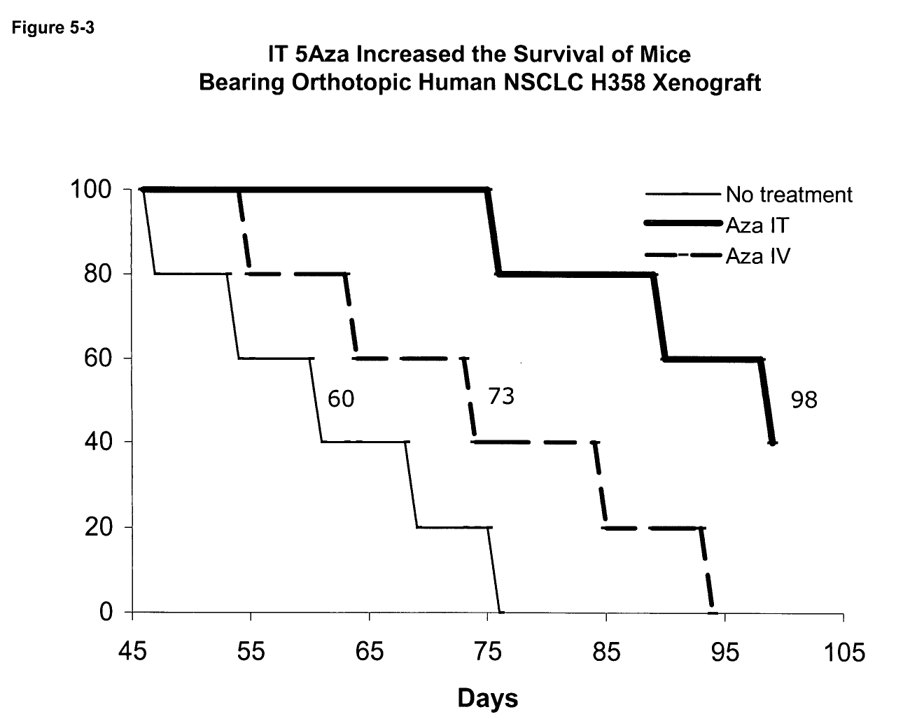

Dose finding. In order to use optimal therapeutic dose, the lethal toxicity and MTD for intratracheal injection (mimicking aerosol, IT) and intravenous injection (mimicking current most frequently used intravenous infusion and injection, IV) routes were determined in mice before the therapeutic experiment. Briefly, the nude mice (female, 6 weeks age) were inoculated with 4×10⁶H460 cells intratracheally. One week later, they were randomly divided into several groups with 5 mice in each. Different groups of mice received different doses of IT 5Aza (range from 2.5 to 50 mg/kg qod×3) or IV 5Aza (range from 6.25 to 50 mg/k daily×6). The percentage of moribund mice in each group versus dose was used to simulate the dose response curves and determined the MTD. The maximum dose caused 0% death was defined as MTD. Under these experimental conditions, the MTD for both IT and IV 5Aza was 37.5 mg/kg or 112.5 mg/m² (total dose, FIG. 5-1). In considering the cytotoxicity results and the aerosol efficiency, 20% of IT MTD and 100% of IV MTD were used for the therapeutic studies.

Antitumor efficacy. In order to evaluate the antitumor efficacy of the airway administered demethylating agent and to compare the result with systemic treatment, orthotopic human NSCLC xenograft models in nude mice were developed by inoculating the tumor cells in the respiratory airway (35,36). In the first experiment, the mice bearing lung tumors were randomly divided into 3 groups with 5 mice in each. Ten days after the H460 tumor inoculation, one group of mice were treated with intratracheal injection of 2.5 mg/kg of 5-azacytidine every other day for 3 injections, another group of mice were treated with IV injections of 6.25 mg/kg every day for 6 injections via tail vein, and the last group of mice remained untreated. In this study, the intratracheal injection of 5-azacytidine showed superior efficacy at only 20% of the IV dose. The median survival is 50, 80 and >109 days for no treatment, IV 5Aza, and IT 5Aza group, respectively (FIG. 5-2). The similar results were obtained from another experiment using an orthotopic H358 NSCLC model. In the second experiment, the treatment schedules and doses were the same as the first experiment. The median survival is 60, 73, and 98 days for no treatment, IV 5Aza, and IT 5Aza group, respectively (FIG. 5-3). In both experiments, the differences between IT 5Aza and IV 5Aza are statistically significant (p<0.01).

### CONCLUSIONS

5-azacytidine was made into an efficient pharmaceutical aerosol formulation that can deliver the drug to lower respiratory airways of human; the aerodynamic size mainly distributed into the optimal range (0.1˜3 μm). Therefore, the present invention shows that 5-azacytidine can demethylate and therefore re-express certain tumor suppressor proteins in NSCLC cell lines.

The present invention further demonstrates that intratracheally injected 5-azacytidine has significantly lower myelotoxicity compared with systemically administered 5-azacytidine. The lung toxicity caused by the intratracheally injected 5-azacytidine at the MDT of IV 5-azacytidine is minimal and reversible.

Furthermore, the present invention demonstrates that intratracheally injected 5-azacytidine is more effective than IV 5Aza against orthotopic human NSCLC xenografts in mice.

### REFERENCES

1. Jemal A, Siegel R, Ward E, Hao Y, Xu J, Murray T, Thu M J. Cancer Statistics, 2008. CA Cancer J Clin, 2008; 58:71-96.
2. Gagnadoux F. Pape A L. Lemarie E. Lerondel S. Valo I. Leblond V. Racineux J L. Urban T. Aerosol delivery of chemotherapy in an orthotopic model of lung cancer. European Respiratory Journal. 26(4):657-61, 2005.
3. Fulzele S V. Shaik M S. Chatterjee A. Singh M. Anti-cancer effect of celecoxib and aerosolized docetaxel against human non-small cell lung cancer cell line, A549. J Pharma Pharmacol. 58(3):327-36, 2006.
4. Otterson G A. Villalona-Calero M A. Sharma S. Kris M G. Imondi A. Gerber M. White D A. Ratain M J. Schiller J H. Sandler A. Kraut M. Mani S. Murren J R. Phase I study of inhaled Doxorubicin for patients with metastatic tumors to the lungs. Clin Cancer Res. 13(4):1246-52, 2007.
5. Wittgen B P. Kunst P W. van der Born K. van Wijk A W. Perkins W. Pilkiewicz F G. Perez-Soler R. Nicholson S. Peters G J. Postmus P E. Phase I study of aerosolized SLIT cisplatin in the treatment of patients with carcinoma of the lung. Clin Cancer Res. 13(8):2414-21, 2007.
6. Zou Y. Tornos C. Qiu X. Lia M. Perez-Soler R. p53 aerosol formulation with low toxicity and high efficiency for early lung cancer treatment. Clin Cancer Res. 13(16):4900-8, 2007.
7. Tsou, J. A., et al., DNA methylation analysis: a powerful new tool for lung cancer diagnosis. Oncogene, 2002. 21(35): 5450-61.
8. Cross, S. H. and A. P. Bird, CpG islands and genes. Curr Opin Genet Dev, 1995. 5(3):309-14.
9. Yen, P. H., et al., Differential methylation of hypoxanthine phosphori-bosyltransferase genes on active and inactive human X chromosomes. Proc Natl Acad Sci USA, 1984. 81(6):1759-63.
10. Ferguson-Smith, A. C., et al., Parental-origin-specific epigenetic modification of the mouse H19 gene. Nature. 1993. 362(6422):751-5.
11. Herman, J. G., et al., Silencing of the VHL tumor-suppressor gene by DNA methylation in renal carcinoma. Proc Natl Acad Sci USA, 1994. 91(21): 9700-4.
12. Merlo, A., et al., 5′ CpG island methylation is associated with transcriptional silencing of the tumour suppressor p16/CDKN2/MTS1 in human cancers. Nat Med, 1995. 1(7): 686-92.
13. Kamb, A., et al., A cell cycle regulator potentially involved in genesis of many tumor types. Science, 1994. 264(5157): 436-40.
14. Otterson, G. A., et al., Absence of p16INK4 protein is restricted to the subset of lung cancer lines that retains wildtype RB. Oncogene, 1994. 9(11): 3375-8.
15. Merlo, A., et al., 5′ CpG island methylation is associated with transcriptional silencing of the tumour suppressor p16/CDKN2/MTS1 in human cancers. Nat Med, 1995. 1(7): 686-92.
16. Palmisano, W. A., et al., Predicting lung cancer by detecting aberrant promoter methylation in sputum. Cancer Res, 2000. 60(21): 5954-8.
17. Deiss, L. P., et al., Identification of a novel serine/threonine kinase and a novel 15-kD protein as potential mediators of the gamma interferon-induced cell death. Genes Dev, 1995. 9(1): 15-30.
18. Cohen, O., E. Feinstein, and A. Kimchi, DAP-kinase is a Ca2+/calmodulin-dependent, cytoskeletal-associated protein kinase, with cell death-inducing functions that depend on its catalytic activity. Embo J, 1997. 16(5):998-1008.
19. Cohen, O., et al., DAP-kinase participates in TNF-alpha- and Fas-induced apoptosis and its function requires the death domain. J Cell Biol, 1999. 146(1): 141-8.
20. Raveh, T. and A. Kimchi, DAP kinase-a proapoptotic gene that functions as a tumor suppressor. Exp Cell Res, 2001. 264(1):185-92.
21. Tang, X., et al., Hypermethylation of the death-associated protein kinase promoter attenuates the sensitivity to TRAIL-induced apoptosis in human non-small cell lung cancer cells. Mol Cancer Res, 2004. 2(12):685-91.
22. Takeichi, M., Cadherin cell adhesion receptors as a morphogenetic regulator. Science, 1991. 251(5000):1451-5.
23. Rosales, C., et al., Signal transduction by cell adhesion receptors. Biochim Biophys Acta, 1995. 1242(1):77-98.
24. Birchmeier, W. and J. Behrens, Cadherin expression in carcinomas: role in the formation of cell junctions and the prevention of invasiveness. Biochim Biophys Acta, 1994. 1198(1):11-26.
25. Bracke, M. E., F. M. Van Roy, and M. M. Mareel, The E-cadherin/catenin complex in invasion and metastasis. Curr Top Microbiol Immunol, 1996. 213 (Pt 1):123-61.
26. Graff, J. R., et al., Distinct patterns of E-cadherin CpG island methylation in papillary, follicular, Hurthle's cell, and poorly differentiated human thyroid carcinoma. Cancer Res, 1998. 58(10):2063-6.
27. Kim, D. S., et al., Aberrant methylation of E-cadherin and H-cadherin genes in nonsmall cell lung cancer and its relation to clinicopathologic features. Cancer, 2007.
28. Christofori, G. and H. Semb, The role of the cell-adhesion molecule E-cadherin as a tumour-suppressor gene. Trends Biochem Sci, 1999. 24(2): 73-6.
29. Kaminskas, E., et al., FDA drug approval summary: azacitidine (5-azacytidine, Vidaza) for injectable suspension. Oncologist, 2005. 10(3): 176-82.
30. Jones, P. A. and S. M. Taylor, Cellular differentiation, cytidine analogs and DNA methylation. Cell, 1980. 20(1):85-93.
31. Juttermann, R., E. Li, and R. Jaenisch, Toxicity of 5-aza-2′-deoxycytidine to mammalian cells is mediated primarily by covalent trapping of DNA methyltransferase rather than DNA demethylation. Proc Natl Acad Sci USA, 1994. 91(25):11797-801.
32. Glover, A. B. and B. Leyland-Jones, Biochemistry of azacitidine: a review. Cancer Treat Rep, 1987. 71(10):959-64.
33. Bates D V, Fish B R, Hatch T F, Mercer T T, Morrow P E. Deposition and retention models for internal dosimetry of the human respiratory tract Task group on lung dynamics. Health Physics 1966; 12:173-207.
34. Davies D S. Pharmacokinetics of inhaled substances. Scandinavian J Respir Disea 1979; 103:44-9.
35. Zou, Y., et al., Effective treatment of early endobronchial cancer with regional administration of liposome-p53 complexes. J Natl Cancer Inst, 1998. 90(15):1130-7.
36. Zou, Y., et al., Development of cationic liposome formulations for intratracheal gene therapy of early lung cancer. Cancer Gene Ther, 2000. 7(5): p. 683-96.
37. Digel, W. and Lubbert, M. DNA methylation disturbances as novel therapeutic target in lung cancer: Preclinical and clinical results. Critical Reviews in Oncology/Hematology, 2005. 55:1-11.
38. Chahinian, A. P., et al. Diffuse pulmonary malignant mesothelioma: response to doxorubicin and 5-azacytidine. Cancer, 1978. 42(4):1687-91.
39. Kiziltepe, T., et al. 5-Azacytidine, a DNA methyltransferase inhibitor, induces ATR-mediated DNA double-strand break responses, apoptosis, and synergistic cytotoxicity with doxorubicin and bortezomib against multiple myeloma cells. Molecular Cancer Therapeutics, 2007. 6:1718-1727.

1. A method for treating bronchial premalignancy or lung cancer in a subject comprising administering to the subject an aerosolized pharmaceutical composition comprising a therapeutically effective amount of 5-azacytidine and a pharmaceutically acceptable carrier.

2. The method of claim 1, wherein dosage of 5-azacytidine is from 2 mg/m² to 600 mg/m².

3. The method of claim 1, wherein dosage of 5-azacytidine is from 5 mg/m² to 200 mg/m².

4. The method of claim 1, wherein dosage of 5-azacytidine is from 7.5 mg/m² to 30 mg/m².

5. The method of claim 1, wherein 5-azacytidine is the only active anticancer agent in the pharmaceutical composition.

6. (canceled)

7. The method of claim 1, wherein the lung cancer is bronchioalveolar carcinoma.

8-11. (canceled)

12. A method for decreasing the likelihood of occurrence of bronchial premalignancy or lung cancer in a subject comprising administering to the subject an aerosolized pharmaceutical composition comprising a prophylactically effective amount of 5-azacytidine and a pharmaceutically acceptable carrier.

13. The method of claim 12, wherein dosage of 5-azacytidine is from 2 mg/m² to 600 mg/m².

14. The method of claim 12, wherein dosage of 5-azacytidine is from 5 mg/m² to 200 mg/m².

15. The method of claim 12, wherein dosage of 5-azacytidine is from 7.5 mg/m² to 30 mg/m².

16. The method of claim 12, wherein 5-azacytidine is the only active anticancer agent in the pharmaceutical composition.

17. (canceled)

18. The method of claim 12, wherein the lung cancer is bronchioalveolar carcinoma.

19-22. (canceled)

23. A pharmaceutical composition for treating bronchial premalignancy or lung cancer or for decreasing the likelihood of occurrence of bronchial premaligancy or lung cancer in a subject comprising a therapeutically or prophylactically effective amount of 5-azacytidine and a pharmaceutically acceptable carrier.

24-44. (canceled)

45. A method for treating bronchial premalignancy and/or lung cancer or decreasing the likelihood of occurrence of bronchial premalignancy and/or lung cancer in a subject comprising administering to the subject an aerosolized pharmaceutical composition comprising a therapeutically or prophylactically effective amount of a demethylating agent and a pharmaceutically acceptable carrier.

46. (canceled)

47. The method of claim 45, wherein the demethylating agent is decitibine.

48. The method of claim 45, wherein the dosage of decitibine is from 0.5 mg/m² to 8000 mg/m².

49. The method of claim 45, wherein the dosage of decitibine is from 1 mg/m² to 5000 mg/m².

50. The method of claim 45, wherein the dosage of decitibine is from 2 mg/m² to 2500 mg/m².
